(19) United States
(12) Patent Application Publication
(10) Pub. No.: US 2010/0185240 A1
(43) Pub. Date: Jul. 22, 2010

(54) BRIDGING DEVICE FOR LAMINOPLASTY AND APPLICATIONS THEREOF

(75) Inventors: Paolo Mangione, Pessac (FR); Jerome Levieux, Geneve (CH)

Correspondence Address: YOUNG & THOMPSON, 209 Madison Street, Suite 500, Alexandria, VA, 22314, US

(73) Assignee: SPINEART SA, Meyrin (CH)

(21) Appl. No.: 12/667,632

(22) PCT Filed: Jul. 1, 2008
(86) PCT No.: PCT/FR08/51217

§ 371 Date: Jan. 4, 2010

(30) Foreign Application Priority Data
Jul. 3, 2007 (FR) 0704800

(51) Int. Cl. A61B 17/70

(52) U.S. Cl. 606/250; 606/277

(57) ABSTRACT

A bridging device for laminoplasty includes a bridge (5) and two attachment arms (7), each attachment arm (7) including a tab ending in a tip and provided with backstop asperities (8), wherein at least one of the attachment arms (7) further includes a blocking device (9) for a cervical blade (2) for clamping the cervical blade (2) between the tab (7) and the blocking device (9), the backstop asperities (8) being provided between the tab (7) and the blocking device (9).

The present invention relates to an artificial cervical lamina and its applications.

Cervical stenosis is a disease which is sometimes operated on surgically. Its main etiology is cervical arthrosis, but many other diseases can result in a significant reduction in the diameter of the spinal canal and cause medullary pain. The object of the intervention in this case is to enlarge the medullary canal.

There exist a number of techniques such as laminectomy, which consists in ablation of the lamina of a cervical vertebra. Another technique is called cervical laminoplasty. In this technique the lamina of a cervical vertebra is opened in order to increase the volume of the canal, and the two parts of the lamina are connected together by a stable and reliable device in order to close the canal again.

In one known device, the two parts of the lamina, having been opened and spread apart, are connected by a bridge, the two pointed ends of which are inserted into the spongy part of the lamina. This technique is suitable where the laminae are thick enough for the bridge to be fitted.

However, there are two major drawbacks to this type of device. When the cervical lamina is very thin, as is frequently the case, the bridge cannot penetrate into the spongy part, which is virtually nonexistent. Furthermore, when the two parts of the cut lamina are separated, one or even both sides may break. In this case, the lamina is no longer retained and laminoplasty is impossible. WO 2005/120367 discloses a length-adjustable laminoplasty bridging device. WO 2003/101319 discloses a bridging device comprising a system of attachment to one part of a lamina.

It would therefore be desirable if there were a multipurpose bridge, suitable for all situations, including where the lamina is very thin and no longer has any spongy region. The device should preferably also enable the lamina to be secured to the vertebra if one or both sides has or have been broken during the opening process.

After long research, the applicant has developed a satisfactory laminoplasty bridging device.

The present application therefore relates to a bridging device for laminoplasty comprising a bridge and two fixing arms, characterized in that each fixing arm comprises a tongue ending in a point and having anti-pullout asperities, and in that at least one of the fixing arms further comprises a cervical lamina locking device for clamping said cervical lamina between the tongue and said locking device, the anti-pullout asperities being located between said tongue and said locking device.

As indicated later the clamping action is provided by elastic deformation.

The overall shape of the bridge and the two fixing arms is usually that of a U.

A bridging device of the invention may comprise a locking device on one side only. Preferably both fixing arms further comprise a cervical lamina locking device for clamping said cervical lamina.

In a preferred implementation of the invention, the above locking device consists of a slat fitted approximately parallel to the tongue and capable of clamping the cervical lamina. The clamping action is very like that of the clip of a pen or a peg. It is achieved by elastic deformation, notably of the first tongue, or of the locking device, or of both.

Advantageously, this slat comprises asperities between said tongue and said slat, facing the anti-pullout asperities provided on the tongue that ends in a point.

Before the locking device is fitted, a gap of for example from 1 to 7 or from 2 to 5 mm may exist between said tongue and said slat. However, it is possible to have no gap between the tongue and the slat and pressure can already be exerted by the latter on each other as a result of their elasticity. The gap is between the ends of the asperities of the tongues and those of the nearest asperities of the slats.

In other preferred ways of implementing the invention, the above locking device consists of a slat curved to follow the vertebral relief and preferably long enough to contain a hole for a screw for fixing into the articular pillar of the vertebra.

The pointed tongues can be inserted into the thickness of the cervical lamina. Also, these tongues have asperities so that they cannot be pulled out after they have been implanted. The locking device, or each locking device, cooperates with the tongue to form a pen clip-like clamp, in order to ensure the stability of the assembly on the lamina.

In the embodiment in which the locking device consists of a curved slat, the holes are advantageously designed to take upward screws (from 30 to 50°).

A laminoplasty bridging device according to the invention can be made in two parts but also by machining a single part forming the whole.

In general, a bridging device according to the invention will be made of metallic material suitable for an implant. It is preferably made of titanium or a titanium alloy but can also be made of an implantable plastic resin such as PEEK. If the bridging device is made of more than one component, they are advantageously all made of the same materials.

Referring to a general U shape, the length of the bridge is advantageously from 8 to 26, preferably from 10 to 24, especially from 12 to 22, and most particularly from 14 to 20 mm.

The length of a tongue is advantageously from 3 to 13, preferably from 4 to 12, especially from 5 to 11 and most particularly from 6 to 10 mm.

The width of a tongue is advantageously from 1 to 10, preferably from 2 to 8, especially from 2 to 6 and most particularly from 3 to 5 mm.

The thickness of a tongue is advantageously from 0.5 to 5, preferably from 0.8 to 4, especially from 1 to 3 and most particularly from 1 to 2 mm.

The dimensions of the slat are advantageously of the same order of magnitude as those of a tongue.

A curved slat will be longer than a tongue and also generally wider at its end to allow for a screwhole.

The asperities may for example be diamond-pointed or grooved.

The bridging devices which form the subject matter of the present invention have very valuable properties and qualities. In particular, they can be fitted easily because of their clamp function, irrespective of whether the tongue is inserted into the thickness of the lamina or is on the inward side of the lamina when the latter is too narrow for the tongue to fit into it. Clamping by elastic deformation as opposed to active fastening avoids the need to use a fastening instrument which would make the surgical process difficult on an often fragile lamina, in an area where there is much danger because it is very close to the spinal marrow. Moreover, if the lamina has been broken on at least one side during the operation, the long curved slat in certain models has holes allowing a screw to be passed through and implanted in the articular pillars, thus allowing the lamina to be reattached to its vertebra.

The bridging devices that form the subject matter of the present invention can be used in the following way. The lamina of the cervical vertebra in question is first opened up. If the laminae are thick enough, the half-laminae are spread apart, and the pointed tongues are implanted into the spongy bone of each half-lamina, which is thus locked between the tongues and the locking device. The canal is thus closed once again.

If the laminae are insufficiently thick, the half-laminae are spread apart, and each half-lamina is sandwiched between the pointed tongue and the locking device. The canal is thus closed once again. In the embodiment in which the locking device consists of a curved second tongue, screws, preferably rising screws, are fitted and fixed into the articular pillar of the vertebra, passing through the holes provided for this purpose. This latter embodiment is especially useful where at least one of the half-laminae has been broken off at its base when the half-laminae were spread apart, since in this case the broken lamina or laminae will no longer hold. The clamp system keeps the half-laminae in position and the device is fastened to the articular pillar, thus stabilizing the whole.

Consequently, the present application also relates to a laminoplasty method in which the procedure described above is performed.

The preferred forms of implementation of the locking devices described above also apply to the other subjects of the invention indicated above, notably to the aforementioned laminoplasty methods.

The invention will be understood more clearly if reference is made to the appended drawings in which

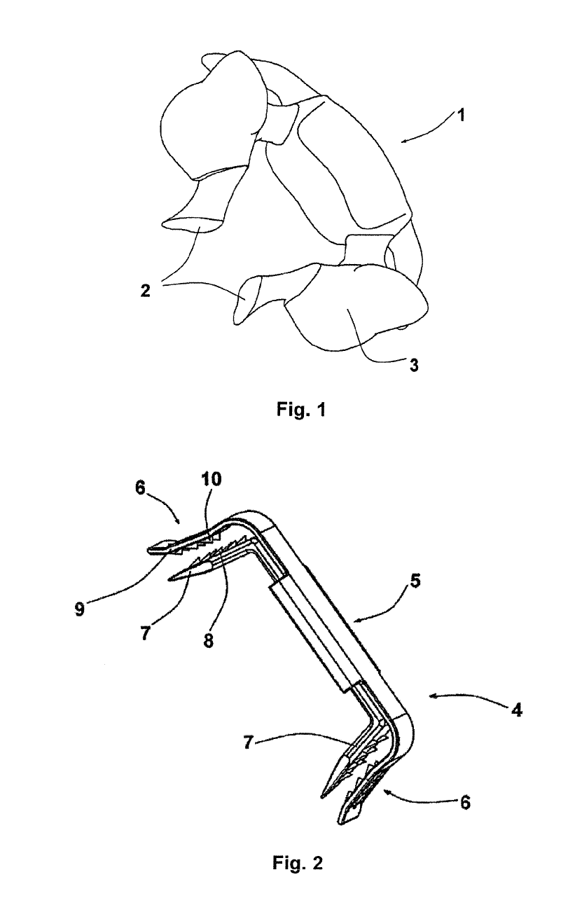

FIG. 1 is a perspective view of a cervical vertebra in which the lamina has been cut

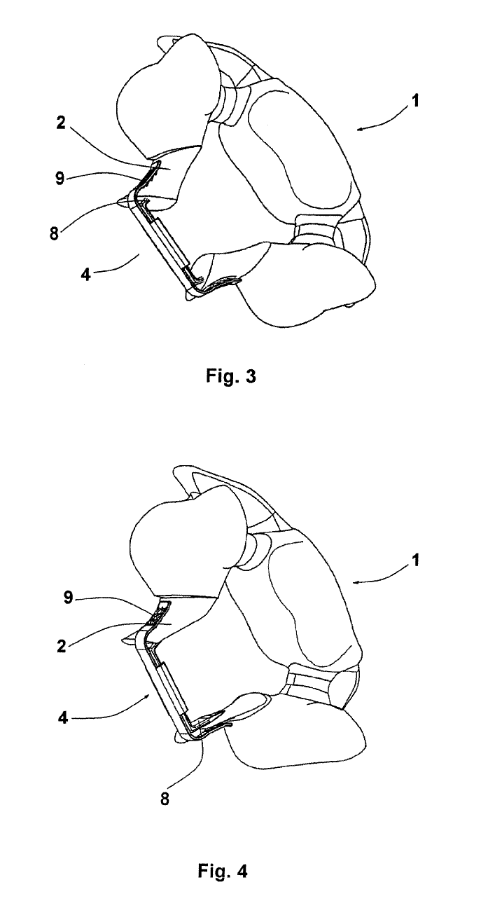

FIG. 2 is a perspective view of a bridging device in a first embodiment

FIGS. 3 and 4 show a bridging device installed on a cervical vertebra in two different fitting arrangements

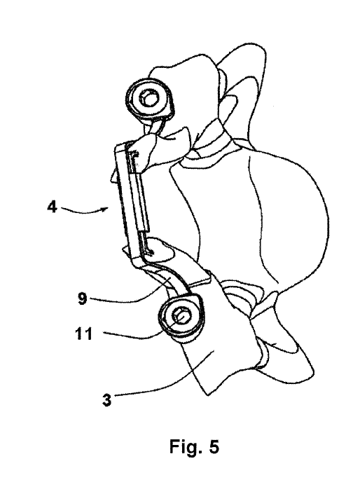

FIG. 5 is a perspective view of a bridging device in a second embodiment in which screws are used for supplementary fixing.

In FIG. 1 a cervical vertebra 1 can be seen in which the lamina 2 connecting the two articular pillars 3 has been cut to enlarge the spinal canal.

FIG. 2 shows a first embodiment of a bridging device 4 according to the invention. That illustrated was made in two separate parts, but a model produced in one part has also been made.

This bridging device 4 basically comprises a generally flat elongate bridge 5, at each end of which is a fixing arm 6 roughly perpendicular to the bridge 5, slightly open but as shown in such a way that the fixing arms 6 are not parallel.

Each of the fixing arms 6 of the model illustrated comprises both a tongue 7 and a locking device 9 which, in this embodiment, is in the shape of a slat.

The tongues 7 are flattened and generally parallelepiped-shaped in cross section and end in a point for ease of insertion into the spongy bone of the lamina 2.

Asperities 8 on the tongues 7 are shaped to produce an anti-pullout effect when the bridging device 4 has been fitted. In the present case the asperities 8 are hook-shaped.

Facing outside of the tongue 7, the locking device 9 is in the present case a slat 9 similar in shape and cross section to the tongue 7 except that it does not end in a point.

The slat 9 also has anti-pullout asperities 10 facing those 8 with which the tongues 7 are provided.

A gap of about 1 mm is provided between the ends of the asperities 8 on the tongues 7 and those of the asperities 10 on the slats 9.

FIG. 3 shows a bridging device 4 according to the invention fitted to a cervical vertebra 1.

The lamina 2 has been cut in its middle and the two half-laminae have been opened to enlarge the canal.

To the extent that the form of the lamina so allows, the tongues 7 have been embedded in the two half-laminae 2.

Half the thickness of each half-lamina 2 is clamped between the tongue 7 and the slat 9. The separation of the tongues 7 and slats 9 away from their natural position of equilibrium results in pressure being applied to the lamina 2. In addition, the anti-pullout asperities 8 and 10 afford an effective grip on the vertebra.

The lamina 2 of the cervical vertebra 1 shown in FIG. 4 is thin.

The fitting shown in FIG. 3 cannot therefore be employed. It is here that the multipurpose nature of the device of the invention is made use of.

The entire thickness of the lamina 2 has been clamped between each tongue 7 and each corresponding slat 9.

FIG. 5 shows a variant. Not only can the lamina or part of the lamina be clamped between the tongue 7 and the slat 9, but also the end of each slat 9 contains a hole 11 for insertion of a screw which can be fixed in the particular pillar 3. A still more effective fixing is thus obtained. It can be seen that the holes are designed to take upward screws (from 30 to 50°).

1-9. (canceled)

10. A bridging device for laminoplasty comprising a bridge (5) and two fixing arms (7), characterized in that each fixing arm (7) comprises a tongue ending in a point and having anti-pullout asperities (8), and in that at least one of the fixing arms (7) further comprises a cervical lamina (2) locking device (9) for clamping said cervical lamina (2) between the tongue (7) and said locking device (9), the anti-pullout asperities (8) being located between said tongue (7) and said locking device (9).

11. The bridging device as claimed in claim 10, characterized in that both fixing arms (7) further comprise a cervical lamina (2) locking device (9) for clamping said cervical lamina.

12. The bridging device as claimed in claim 10, characterized in that the locking device consists of a slat (9) fitted approximately parallel to the tongue (7) and capable of clamping the cervical lamina (2).

13. The bridging device as claimed in claim 12, characterized in that the slat (9) comprises asperities (10) between the tongue (7) and the slat (9), facing the anti-pullout asperities (8) provided on the tongue that ends in a point.

14. The bridging device as claimed in claim 10, characterized in that a gap of 1 to 7 mm is provided between the tongue (7) and the slat (9).

15. The bridging device as claimed in claim 10, characterized in that the locking device (9) consists of a slat (9) curved to follow the vertebral relief.

16. The bridging device as claimed in claim 15, characterized in that the curved slat (9) is long enough to contain a hole for a screw for fixing into the articular pillar (3) of the vertebra.

17. The bridging device as claimed in claim 10, characterized in that it is made in two parts or in one part.

18. The bridging device as claimed in claim 10, characterized in that its general shape is that of a U, in which the length of the bridge (5) is from 10 to 24 mm, the length of a tongue (7) is from 4 to 12 mm, the width of a tongue (7) is from 2 to 8 mm, and the thickness of a tongue (7) is from 0.8 to 4 mm.

19. The bridging device as claimed in claim 11, characterized in that the locking device consists of a slat (9) fitted approximately parallel to the tongue (7) and capable of clamping the cervical lamina (2).
